(19) United States
(12) Patent Application Publication
(10) Pub. No.: US 2015/0182700 A1
(43) Pub. Date: Jul. 2, 2015

(54) PRESSURE-SENSING AUTOMATIC INJECTION SYSTEM

(71) Applicant: PANACE CO., LTD., Seongnam-si (KR)

(72) Inventor: Si-Yeol Bang, Yongin-si (KR)

(73) Assignee: PANACE CO., LTD., Seongnam-si (KR)

(21) Appl. No.: 14/406,063

(22) PCT Filed: Jun. 3, 2013
(86) PCT No.: PCT/KR2013/004865

§ 371 Date: Dec. 5, 2014

(30) Foreign Application Priority Data
Jun. 21, 2012 (KR) 10-2012-0066699

(51) Int. Cl. A61M 5/20

(52) U.S. Cl. CPC A61M 5/20; A61M 2205/3344; A61M 5/3298

(57) ABSTRACT

Disclosed is a system that senses the pressure of a suction cap and automatically performs injection. The present invention comprises: a suction motor for increasing the suction pressure to the suction cap of a suction injection needle; a solenoid valve having a solenoid valve controller connected thereto and having the function of relieving the suction pressure of the suction cap; a pressure sensor having a drug injection controller connected thereto and measuring the pressure of the suction cap; a drug injection motor operated by the drug injection controller on the basis of the pressure of the suction injection needle measured by the pressure sensor; a screw gear bolt connected to a syringe piston pusher which pushes a syringe piston along the lengthwise direction of a drug injection supporting portion; a screw gear shaft passing through the screw gear bolt and rotating according to the operation of the drug injection motor; and a connecting member for interconnecting the solenoid valve, the suction motor, the pressure sensor, and the suction injection needle. Thus, after the suction cap is attached to the skin, the pressure is sensed and a set amount of drugs is automatically injected. Therefore, the accuracy of drug treatment can be increased.

### BACKGROUND OF THE INVENTION

1. Field of the Invention

The present invention relates to an automatic injection system having one or more injection needles operable in response to sensing pressure, and more particularly, to an automatic injection system which senses pressure between a practitioner and an automatic injection apparatus and automatically and conveniently injects medication.

2. Description of the Related Art

Since Mesotherapy was introduced by a French doctor (Dr. Michel Pistor) in 1952, it has been widely practiced in Europe and South America for treatment purposes. The Mesotherapy procedure generally involves use of a special-purpose injection equipment and micro-thin syringe, by which a small amount of treatment medicine is injected into a skin layer and subcutaneous layer. The ailments that are subject of Mesotherapy include acute/chronic pains, partial obesity, and ailments that require cosmetic/plastic surgical treatments. Mesotherapy has recently been very popular for its instant effect of promoting blood circulation and lymph circulation at the site of injection, removing various pains and partial obesities and wrinkles, and preventing skin aging and enhance skin elasticity, with pain-free procedure and almost no adverse side effects. For a practitioner of Mesotherapy, it is important to elaborately adjust, with the tips of the fingers, the thickness of needle, depth and angle of injection, amount of medication being shoot and site of treatment. Generally, because the practitioner fills a disposable syringe with a predetermined amount of medication and injects the medication at the site of treatment by hands, it takes time and procedure takes long, and the procedure becomes considerably inconvenience due to increasing fatigue. Accordingly, a mesogun or an injection gun is used as a means for filling medications.

Having medication delivered into skin by use of a needle is a very effective system and it has been widely used in the modern treatment procedures. However, one single needle can be very inconvenient to use, when it is necessary to treat a relatively wide area of skin, scalp, etc. Accordingly, a sterilized multi-needle having a plurality of needles has been developed to address the inconveniences mentioned above. However, skin treatment using multi-needles equipped at 1 to 1.5 mm intervals can be disadvantageous, particularly because the needles often pushes against the skin, instead of being properly inserted into skin.

### DETAILED DESCRIPTION OF THE INVENTION

### Technical Object

A single needle can be less efficient when it is necessary to treat a wide area of skin, scalp, etc. Although a multi-needle having a plurality of needles thereon has been developed and used to resolve the inconvenience, the skin treatment using multi-needle is frequently accompanied with incomplete insertion of the needles because the needles push against the skin instead of being inserted. Accordingly, an apparatus and a method are necessary, which can properly insert a needle into skin and accurately inject medication.

### Means to Solve the Object

According to an embodiment of the present invention, a pressure-sensing automatic injection system is disclosed, which includes a suction motor for increasing the suction pressure at a suction cap of a suction injection needle, a solenoid valve having a solenoid valve controller connected thereto and having the function of relieving the suction pressure of the suction cap, a pressure sensor having a drug injection controller connected thereto and measuring the pressure of the suction cap, a drug injection motor operated by the drug injection controller on the basis of the pressure of the suction injection needle measured by the pressure sensor, a screw gear bolt connected to a syringe piston pusher which pushes a syringe piston along the lengthwise direction of a drug injection supporting portion, a screw gear shaft passing through the screw gear bolt and rotating according to the operation of the drug injection motor, and a connecting member for interconnecting the solenoid valve, the suction motor, the pressure sensor, and the suction injection needle.

### Effect of the Invention

According to an embodiment of the present invention explained herein, the pressure-sensing automatic injection apparatus senses suction pressure so that when the suction pressure exceeds a predetermined level, the pressure-sensing automatic injection apparatus determines that a suction cap head of suction injection needles is in close contact with the skin, with the needles being in inserted position into the skin, and thus automatically allows medication to be injected. The apparatus is a system that automates a sequence of procedures including, upon completion of the injection, stopping a suction motor so that the suction injection needles are naturally separated from the skin, opening the solenoid valve to relieve the pressure of the suction cap, giving off a signaling sound to let the practitioner to move the suction injection needles to another site for treatment.

With this apparatus, the practitioner does not have to do the medication injecting action such as pressing a hand switch or foot switch during each procedure, but can instead rely on the automatic injection system for such action. Accordingly, the practitioner can pay more attention to his/her own role of determining an exact location for treatment.

### BRIEF DESCRIPTION OF THE DRAWINGS

FIG. 1 is a diagram of a pressure-sensing automatic injection system according to an embodiment of the present invention.

FIG. 2 is a perspective view of a pressure-sensing automatic injection system according to an embodiment of the present invention.

### BEST MODE

According to an embodiment of the present invention, a pressure-sensing automatic injection system is disclosed, which includes a suction motor for increasing the suction pressure at a suction cap of a suction injection needle, a solenoid valve having a solenoid valve controller connected thereto and having the function of relieving the suction pressure of the suction cap, a pressure sensor having a drug injection controller connected thereto and measuring the pressure of the suction cap, a drug injection motor operated by the drug injection controller on the basis of the pressure of the suction injection needle measured by the pressure sensor, a screw gear bolt connected to a syringe piston pusher which pushes a syringe piston along the lengthwise direction of a drug injection supporting portion, a screw gear shaft passing through the screw gear bolt and rotating according to the operation of the drug injection motor, and a connecting member for interconnecting the solenoid valve, the suction motor, the pressure sensor, and the suction injection needle.

[Mode for Carrying Out the Invention]

The preferred embodiments of the present invention will be explained below with reference to accompanied drawings. While the invention can have a variety of modifications and configurations, certain embodiments have been illustrated and explained herein. However, this should not be construed as limiting the invention to any specific disclosed configurations, but rather understood as including all the modifications, equivalents or replacements that may be included under concept and technical scope of the present invention.

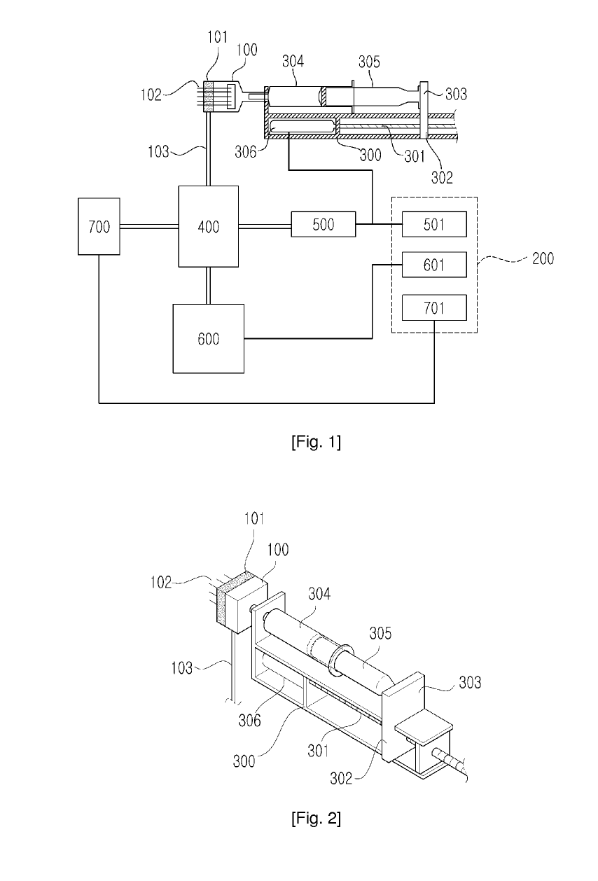

FIG. 1 is a diagram of a pressure-sensing automatic injection system according to an embodiment of the present invention, and FIG. 2 is a perspective view of a pressure-sensing automatic injection system according to an embodiment of the present invention.

As illustrated in FIG. 1, the pressure-sensing automatic injection system according to an embodiment of the present invention includes a suction injection needle 100, a drug injection supporting portion 300, a drug injection motor 306, a screw gear shaft 301, a screw gear bolt 302, a syringe piston pusher 303, a solenoid valve 700, a pressure sensor 500, a suction motor 600, a suction air tube 103, a drug injection controller 501, a suction motor controller 601 and a solenoid vale controller 701.

The suction motor 600 sucks out the internal air of the suction injection needle 100, and when the suction cap 101 of the suction injection needle 100 is in contact with a patient, the front end of the suction injection needle is blocked by the skin, so that the pressure of the suction injection needle is increased. The suction motor 600 is connected to the suction cap 102 via the connecting member 400 and the suction air tube 103, in which the connecting member 400 is connected also to the solenoid valve 700 and the pressure sensor 500. The suction motor is connected in parallel to the suction motor controller 601 of an electronic control device 200 of the present system, and the suction motor controller 601 controls the pressure and time of suction by the suction motor. The suction motor increases effect of the suction injection needle 100 being brought into tight contact with the site of treatment, before administration of a predetermined amount of medication by the drug injection motor 306 (will be explained below). Furthermore, the suction motor 600 also has a function of injecting the multi-needles 102 stably into the site of treatment of the patient.

The suction injection needle 100 has the multi-needles 102 at the center, and the suction cap 101 provided at a location where the suction injection needle meets skin of a patient. The suction injection needle 100 is connected to a front end of the drug injection supporting portion 300. The multi-needles 102 each has a hollow cylindrical body, whose rear end is connected to the suction injection needle 100. The multi-needles 102 play a role of introducing drug sprayed from the syringe attached to the drug injection supporting portion 300 onto a patient. The suction cap 101 is connected to the connecting member 400 via the suction air tube 103, in which the connecting member 400 is connected to the solenoid valve, the suction motor and the pressure sensor. As the practitioner places the suction injection needle on the skin of the patient, the pressure inside the suction injection needle increases due to the suction motor, and accordingly, the multi-needles are inserted into the skin of the patient. The depth to which the multi-needles are injected may vary depending on the size of the suction injection needles, pressure set at the suction motor, or type of material that constitutes the suction cap. For example, an elastic member such as rubber or sponge may be used. The practitioner may set the pressure of the suction motor in advance to adjust the depth the multi-needles are inserted into the patient.

The drug injection supporting portion 300 includes the drug injection motor 306, the screw gear shaft 301, the screw gear bolt 302 and the syringe piston pusher 303, and is attached with a syringe with drug filled therein. When the pressure sensor measures the pressure of the suction cap to be exceeding a set pressure, a microcontroller of the electronic control apparatus 200 determines that the suction cap is in close contact with the skin of the patient, and thus operates the drug injection motor 306 via the drug injection controller 501. As the drug injection motor 306 is operated, the screw gear shaft 301 connected to the drug injection motor 306 is rotated, and the screw gear bolt 302 geared with the screw injection motor 306 is moved by screw-rotation along a lengthwise direction of the drug injection supporting portion 300. The screw gear bolt 302 is connected to the syringe piston pusher 303 which exerts pressure onto the syringe piston 305. The syringe piston pusher 303 starts to push the syringe piston 305 according to the motion of the screw gear bolt 302, and when the drug injection motor 306 is moved as previously set, a preset amount of the drug is administered from the syringe into the patient.

When the preset amount of drug is injected, the drug injection controller 501 stops rotation of the drug injection motor. After that, the electronic control apparatus 200 is operated to cause the suction injection needle 100 to separate from the contact with the skin of the patient, in which the suction motor controller 601 stops the suction motor 600 and the solenoid valve controller 701 opens the solenoid valve 700 to relieve the increased pressure at the suction cap 101. When the suction pressure at the suction cap 101 is relieved, the suction syringe needle is naturally separated from the skin, by then the practitioner can take steps for the next procedure. Additionally, the electronic control apparatus may include an alarm generator which may generate a signaling sound to notify it, once administering of a single dose of the drug as previously set is completed. After administering a single dose of drug as previously set, the practitioner may move the suction injection needle to another site on the skin of the patient and continue injecting a preset amount of drug. The system explained above enables the practitioner to elaborately proceed the treatment and focus on the site of treatment, as the system does not rely on empirical decision of the practitioner in injecting the drug.

### INDUSTRIAL APPLICABILITY

The invention relates to a system which senses pressure at a suction cap and automatically injects medication, and according to the present invention, the automatic injection system senses the pressure when the suction cap is attached onto skin of the patient, and automatically injects a preset dose of medication, and is applicable in medical and cosmetic field.

1. A pressure-sensing automatic injection system, comprising:
a suction motor for increasing a suction pressure at a suction cap of a suction injection needle;
a solenoid valve having a solenoid valve controller connected thereto and having a function of relieving the suction pressure of the suction cap;
a pressure sensor having a drug injection controller connected thereto and measuring the pressure of the suction cap;
a drug injection motor operated by a drug injection controller on the basis of the pressure of the suction injection needle as measured by the pressure sensor;
a screw gear bolt connected to a syringe piston pusher which pushes a syringe piston along a lengthwise direction of a drug injection supporting portion;
a screw gear shaft passing through the screw gear bolt and rotating according to the operation of the drug injection motor; and
a connecting member for interconnecting the solenoid valve, the suction motor, the pressure sensor, and the suction injection needle.

2. The pressure-sensing automatic injection system of claim 1, wherein the suction injection needle comprises multi-needles.

3. The pressure-sensing automatic injection system of claim 1, wherein the connecting member is connected to the solenoid valve, the pressure sensor, the suction motor, and the suction injection needle with a tube, the tube being formed from an elastic member.

4. A pressure-sensing automatic injection system, comprising:
a suction motor for increasing a suction pressure at a suction cap of a suction injection needle;
a solenoid valve having a solenoid valve controller connected thereto and having a function of relieving the suction pressure of the suction cap;
a pressure sensor having a drug injection controller connected thereto and measuring the pressure of the suction cap;
a drug injection motor operated by a drug injection controller on the basis of the pressure of the suction injection needle as measured by the pressure sensor;
a screw gear bolt connected to a syringe piston pusher which pushes a syringe piston along a lengthwise direction of a drug injection supporting portion;
a screw gear shaft passing through the screw gear bolt and rotating according to the operation of the drug injection motor;
a connecting member for interconnecting the solenoid valve, the suction motor, the pressure sensor, and the suction injection needle; and
a signaling system for generating a signal upon determination by the pressure sensor a relief of the suction pressure of the suction cap.

5. The pressure-sensing automatic injection system of claim 4, wherein the suction injection needle comprises multi-needles.

6. The pressure-sensing automatic injection system of claim 4, wherein the connecting member is connected to the solenoid valve, the pressure sensor, the suction motor, and the suction injection needle with a tube, the tube being formed from an elastic member.

7. A pressure-sensing automatic injection method comprising:
(a) at a suction motor controller, exerting a suction pressure around a suction cap of a suction injection needle, by operating a suction motor;
(b) contacting a portion of the suction cap, which is a front end of the suction injection needle, onto a site for injection on a skin, and increasing the suction pressure of the suction cap;
(c) at a pressure sensor, measuring the pressure of the suction cap via a connecting member and driving a drug injection motor connected to the pressure sensor upon reaching a preset pressure;
(d) at a screw gear bolt through which a screw gear shaft is passed, pushing a syringe piston in accordance with an operation of a drug injection motor connected to the screw gear shaft;
(e) injecting drug stored in a syringe through the suction injection needle, under pressure exerted by the syringe piston;
(f) stopping the operation of the drug injection motor and stopping the suction motor; and
(g) relieving the suction pressure of the suction cap by opening a solenoid valve.

8. The pressure-sensing automatic injection method of claim 7, wherein, when the drug injection motor is operated, the drug injection controller controls the drug injection motor to inject a preset amount of drug.

9. The pressure-sensing automatic injection method of claim 7, further comprising generating a signaling sound for notification of a practitioner, upon relief of the suction pressure of the suction cap.

10. The pressure-sensing automatic injection system of claim 2, wherein the connecting member is connected to the solenoid valve, the pressure sensor, the suction motor, and the suction injection needle with a tube, the tube being formed from an elastic member.

11. The pressure-sensing automatic injection system of claim 5, wherein the connecting member is connected to the solenoid valve, the pressure sensor, the suction motor, and the suction injection needle with a tube, the tube being formed from an elastic member.

12. The pressure-sensing automatic injection method of claim 8, further comprising generating a signaling sound for notification of a practitioner, upon relief of the suction pressure of the suction cap.
